(19) United States
(12) Patent Application Publication
(10) Pub. No.: US 2004/0127878 A1
(43) Pub. Date: Jul. 1, 2004

(54) Surround stretch absorbent garments

(76) Inventors: Christopher Peter Olson, Neenah, WI (US); Yee Yang, Appleton, WI (US)

Correspondence Address: KIMBERLY-CLARK WORLDWIDE, INC., 401 NORTH LAKE STREET, NEENAH, WI, 54956

(21) Appl. No.: 10/331,896

(22) Filed: Dec. 30, 2002

(51) Int. Cl. A61F 13/15; A61F 13/20

(52) U.S. Cl. 604/385.16

(57) ABSTRACT

A pant-like absorbent article, such as a child's training pant, includes laterally extending front and back ear portions defined at opposed lateral sides of front and back waist regions. An absorbent body structure extends longitudinally along the crotch region between at least a partially stretchable outercover and at least a partially stretchable liner, from the front waist region to the back waist region, the absorbent body structure including laterally extending wing portions that extend laterally along the front and back ear portions. The laterally extending wing portions are stretchable and absorbent, to allow for the transfer of liquid from the front to the back of the article while a user is in a reclining or sleeping position.

### TECHNICAL FIELD OF THE INVENTION

The present invention relates generally to the field of absorbent articles and garments, such as disposable diapers, child's training pants, incontinence articles, and the like, which incorporate stretch absorbent materials into their structure.

### BACKGROUND

Many types of consumer products such as disposable diapers, training pants, feminine care articles, incontinence articles, and the like, utilize an absorbent pad structure (retention layer) for absorbing and wicking away bodily liquids. The absorbent pads are conventionally formed from an absorbent web, typically a non-woven fibrous web material formed by known techniques and are disposed between a liquid pervious topsheet or bodyside liner layer and a liquid impermeable backsheet or outercover layer. Such absorbent articles may also include an elasticized waistband and leg cuffs to help reduce the leakage of body exudates and to provide improved fit to the consumer. Some conventional absorbent articles have also included elasticized containment or barrier flaps at the leg or waist sections of the article to further reduce the occurrence of leaks.

Conventional pant-like absorbent structures, such as a child's training pant, have also incorporated stretch portions along their sides such as in the garment hip areas. Reference is made, for example, to the HUGGIES® PULL-UPS® disposable training pants from Kimberly-Clark Corporation of Neenah, Wis. The use of such elastic side panels in training pants is also described, for example, in U.S. Pat. No. 4,940,464 and WO 037009 which are incorporated by reference hereto in their entirety. Such conventional training pants include elastomeric side panels that are joined at side seams extending between a waist opening and respective leg openings. The seams may be permanent wherein the article is pulled onto the wearer in a manner similar to underwear. Alternatively, the seams may be releasable wherein the article may be put on and/or taken off similar to a disposable diaper. However, with these types of configurations, it is not uncommon for leakage to occur when users are reclining or lying down, and particularly, when they are sleeping on their sides. With conventional absorbent articles, the absorbent body layer does not extend around the hips between the abdomen and the buttocks and may be insufficient to prevent leakage, because any overflow of urine (especially for males) when the wearer is lying on the side can go directly from the front of the garment via gravity to the side area of the garment, and then ultimately, leak out if there is no absorbent in the garment side areas.

There is therefore a need for an improved absorbent pad structure and stretch configuration that will help minimize the occurrence of side area leaks, in and around the hip areas, particularly in training pant articles, where leakage may result from a user sleeping or lying on his/her sides.

### SUMMARY OF THE INVENTION

In general, the present invention provides an improved absorbent article that protects against leakage while the wearer is lying on the side. A particularly useful embodiment of the invention is a child's training pant. However, other absorbent articles such as incontinence devices, disposable diapers, and the like, are also within the scope and spirit of the invention.

A pant-like absorbent article includes a chassis defining a front waist region at a first longitudinal end, a back waist region at an opposite longitudinal end, and a crotch region extending longitudinally between the front and back waist regions. The chassis includes at least a topsheet, an outercover, and an absorbent body structure including a central portion and laterally extending wing portions. The chassis may be composed generally entirely of stretchable and desirably elastomeric materials, or may alternatively only have particular regions or portions made up of stretchable and desirably elastomeric materials. In any event, such chassis includes at least a partially stretchable and desirably elastomeric outercover, at least a partially stretchable and desirably elastomeric topsheet or liner layer, and side absorbent and elastomeric combination materials adjacent/proximate either the side seam or refastenable/releasable and reattachable components along the laterally extending wing portions of the absorbent body structure, in order to allow liquid to pass from the front side to the back side of the absorbent article, while the article is being worn by a user in a reclining or sleeping position. Essentially, when the garment is being worn, the side elastomeric and absorbent materials will be proximate to each other so as to allow liquid to pass from the front side to the back side of the article. Such a configuration provides for both the stretch that user's have grown accustomed to in absorbent articles, but also provides additional leakage protection. It should be appreciated that the materials used for the article are not a limiting factor, and that many combinations of materials and compositions may be used in the manufacture of articles according to the invention.

In a typical training pant configuration, laterally extending ear portions are defined at opposed lateral sides of one or both of the front and back waist regions. These ear portions extend around the front and back waist of a wearer and meet at side seams or refastenable components of the article. The ear portions also define laterally inwardly directed leg contours. When the chassis is formed into the pant-like article, the ear portions are joined at the side seams (or refastenable portions) and the leg contours define leg openings in conjunction with the crotch region of the article.

As previously indicated, the absorbent body structure has a central portion that extends longitudinally along the crotch region generally from the front waist region to the back waist region and includes laterally extending wing portions that extend laterally along the front and/or back ear portions of the chassis. These laterally extending wing portions may be a physically contiguous part of the central portion. Such laterally extending wing portions may be of similar thickness or basis weight to the central portion, or alternatively, may be of different thickness or basis weight. Alternatively, the laterally extending wing portions may be physically separate portions adjacent and proximate to the central portion, but in liquid communication with the central portion. As an alternative embodiment, either the central portion of the absorbent body structure may overlap the edges of the separate laterally extending wing portions of the absorbent, or vice versa, such that the overlap occurs in the Z direction through the product. The central portion of the absorbent body structure may include any combination of conventional absorbent materials used in absorbent articles. As an alternative, it may be comprised of a material that demonstrates both absorbency and stretch functions. As a further alternative, it may be comprised of layered absorbent materials in the Z direction, such as traditional high capacity absorbent materials in a first layer closest to the outercover and a lower capacity absorbent in a second layer closest to the topsheet, but with the second layer also including stretch functionality. The utility of layering is that it can enable the garment to have particularly advantageous combinations of stretch functionality and leakage protection. The elastomeric materials used in stretch absorbents displace absorbent materials. As such, increasing the elastic properties of an absorbent can reduce its absorbent capacity, up-take rate or permeability. Therefore stretch absorbents used in garments like those described herein, may in some alternative embodiments be, a compromise between absorbency and stretch characteristics, and cost, which layering can facilitate.

In still a further alternative, the laterally extending wing portions of the absorbent body structure may include stretch and absorbent materials, without stretch absorbent materials being present in the central absorbent portion. This structure can be beneficial, in that absorbency is enhance where the urine or other exudates are concentrated in the garment front areas, and stretch is enhanced in the side areas of the garment, where it is important to facilitate the donning of the garment.

The total capacity of the mixture of stretchable materials and absorbent materials is desirably considered when determining ultimate garment configuration. The capacity differences previously described can be due to either differences in the actual absorbent materials used or to differences in the amount of absorbent materials, resulting from absorbent material displacement by higher amounts of elastomer or to a combination of these effects. Therefore, in still further alternative embodiments, the absorbent structure material in the various portions may be varied by either absorbent material type or absorbent material content, in order to create relative differences in absorbent capacity. For instance, in an alternative embodiment, an absorbent article includes a central portion and laterally extending wing portions, with relatively higher capacity absorbent structure in the central portion, and laterally extending wing portions including lower capacity absorbent structure relative to the absorbent structure in the central portion.

In a further alternative embodiment, it would be desirable to place relatively higher absorbent capacity material in the wings and lower absorbent capacity materials in the central portion. This would be desirable in order to compensate for the displacement of the absorbent materials from the higher amounts of elastomeric materials in the laterally extending wings. It would also offer an alternative if it was desired to provide absorbent structure performance of the wings to be closer to that of the central portion.

In still a further alternative embodiment, the article has a central portion with a superabsorbent material having a gram/gram absorbent capacity that is at least 10% greater than a superabsorbent material in said laterally extending wing portions.

In a useful embodiment, at least the front and back ear portions of the chassis waist regions, including the absorbent article structure, are stretchable. For example, essentially the entire chassis may be stretchable, such that the liner, all portions of the absorbent body structure, and the outer cover are stretchable. In still an additional embodiment, the chassis may include “bridge”-like materials in the laterally extending side wing portions of the absorbent body structure, so as to allow the passage of liquid from front to back of the article, when the user is in a reclining or sleeping position. Such “bridge”-like materials, would provide the beneficial liquid communication and could either have high or relatively low amounts of stretch functionality depending on the design needs of the particular garment product.

In additional alternative embodiments, the absorbent article may include various types of side seam configurations. For example, the ear portions may be bonded at the side seams such that the wearer must pull the article on much like underwear. These bonded seams may also be tearable such that the article may be removed from the wearer by separating or tearing the seams and removing the article much like a diaper. In an alternate embodiment, the side seams are releasable and re-attachable. For example, a hook-and-loop, or other type of re-attachable system may be used along the side seams. In still a further alternative embodiment, the side seams are bonded at different depths in order to provide a water impervious seam.

Aspects of the invention will be described below in greater detail with reference to embodiments shown in the figures.

### BRIEF DESCRIPTION OF THE FIGURES

FIG. 1 is a front perspective view of a representative absorbent article, in this case a child's training pant.

FIG. 2 is a simplified topside (bodyside) plan view of the absorbent article chassis of FIG. 1 in a flat out state (without the gathering normally associated with an article with elastomeric components).

FIG. 3 is a schematic (showing the Z direction) cross-sectional exploded view of the chassis taken of FIG. 2 along the lines 3-3 indicated in FIG. 2.

FIG. 4 is a cross-sectional view along line A-A (from FIG. 1) of a pant chassis similar to that of FIG. 1, that is representative of an article that is the invention.

FIG. 5 is a close-up cross-sectional view of an alternative embodiment of the circled side seam area labeled B in FIG. 4.

FIG. 6 is a close-up cross-sectional view of an alternative embodiment of the circled side seam area labeled B of FIG. 4.

FIG. 7 is a cross-sectional view of an alternative embodiment of a pant chassis similar to that of FIG. 1.

FIG. 8 is a close-up cross-sectional view of the circled side seam area labeled C of FIG. 7.

FIG. 9 is a cross-sectional view of an alternative embodiment of a pant chassis similar to that of FIG. 7.

FIG. 10 likewise, is a cross-sectional view of an alternative embodiment of a pant chassis similar to that of FIG. 7.

FIG. 11 is a close-up cross-sectional view of the circled side area labeled D of FIG. 10.

FIG. 12 is a close-up cross-sectional view of the circled side seam area labeled B of FIG. 4.

FIGS. 13-15 are several views of a Saturated Capacity Tester Vacuum Apparatus.

### DETAILED DESCRIPTION

The invention will now be described in detail with reference to particular embodiments thereof. The embodiments are provided by way of explanation of the invention, and are not meant as a limitation of the invention. For example, features described or illustrated as part of one embodiment may be used with another embodiment to yield still a further embodiment. It is intended that the present invention include these and other modifications and variations as come within the scope and spirit of the invention.

Definitions

Within the context of the present description, the following terms may have the following meanings:

As used herein, the term “absorbent article” shall be synonymous with “personal care product” and shall mean diapers, training pants, absorbent underpants, adult incontinence products, bandages, veterinary, mortuary and feminine care/hygiene products.

As used herein the term “absorbent capacity” shall be synonymous with saturated capacity as measured by the “Saturated Capacity Test Method” described herein.

The term “absorbent materials” refer to the materials that are included in the absorbent structures that serve the function of absorbing and then containing urine, moisture or other body exudates.

The terms “absorbent structure”, “absorbent material structure” and “absorbent body structures” shall be used synonymously and refer to a layer in the article that contains the absorbent materials. It may also contain elastomeric materials.

As used herein, the term “airlaying” shall have the same meaning as “airlaid” and is a well known process by which a fibrous nonwoven layer can be formed. In the airlaying process, bundles of small fibers having typical lengths ranging from about 6 to about 19 millimeters (mm) are separated and entrained in an air supply and then deposited onto a forming screen, usually with the assistance of a vacuum supply. The randomly deposited fibers then are bonded to one another using, for example, hot air or a spray adhesive. The absorbent composites of this invention may be made using the airlaid process. The production of airlaid nonwoven composites is well defined in the literature and documented in the art. Examples include the DanWeb process as described in U.S. Pat. No. 4,640,810 to Laursen et al. and assigned to Scan Web of North America Inc, the Kroyer process as described in U.S. Pat. No. 4,494,278 to Kroyer et al. and U.S. Pat. No. 5,527,171 to Soerensen assigned to Niro Separation a/s, the method of U.S. Pat. No. 4,375,448 to Appel et al. assigned to Kimberly-Clark Corporation, or other similar methods. Each of the foregoing patents is hereby incorporated by reference in its entirety.

“Attached” refers to the bonding, joining, adhering, connecting, attaching, or the like, of two elements. Two elements will be considered to be attached together when they are bonded directly to one another or indirectly to one another, such as when each is directly attached to an intermediate element.

As used herein, the tem “bonded carded web” refers to webs that are made from staple fibers which are sent through a combing or carding unit, which breaks apart and aligns the staple fibers in the machine direction to form a generally machine direction-oriented fibrous nonwoven web. Such fibers are usually purchased in bales which are placed in a picker which separates the fibers prior to the carding unit. Once the web is formed, it then is bonded by one or more of several known bonding methods. One such bonding method is powder bonding, wherein a powdered adhesive is distributed through the web and then activated, usually by heating the web and adhesive with hot air. Another suitable bonding method is pattern bonding, wherein heated calender rolls or ultrasonic bonding equipment are used to bond the fibers together, usually in a localized bond pattern, throughout the web. The web can also be bonded across its entire surface if so desired. Another suitable and well-known bonding method, particularly when using bicomponent staple fibers, or other adhesive fibers, is through-air bonding.

As used herein and in the claims, the term “comprising” is inclusive or open-ended and does not exclude additional unrecited elements, compositional components, or method steps. “Elastomeric” refers to a material or composite which can be stretched or elongated by at least 25% of its relaxed length in at least one direction, and which will recover, upon release of the applied force, at least 10% of its elongation. It is generally preferred that the elastomeric material or composite be capable of being elongated by at least 100%, more desirably by at least 300%, of its relaxed length and recover at least 30% and more desirably 50% of its elongation upon release of a stretching, biasing force, within about one minute. An elastomeric material may thus be described as stretchable and “stretchy”.

For the purposes of this application the terms “stretchy”, “stretchable”, “extensible”, “elongatable” and “stretch” may be used interchangeably and shall describe a material function/attribute in which a given material is capable of elongating/extending or stretching in at least one direction a certain elongation, upon application of a biasing force (for instance, a biasing force in a range of 80 to 800 gm per linear cm). For instance, an extensible material can be stretched without breaking by at least 25% (to at least 125% of its initial unstretched length) in at least one direction, suitably by at least 50% (to at least 150% of its initial unstretched length), more suitably by at least 100% (to at least 200% of its initial unstretched length). As an example, an extensible material having an initial unstretched length of 3 inches (7.6 centimeters) may be stretched without breaking to at stretched length of at least 3.75 inches (9.5 centimeters) in at least one direction (for the “by at least 25%” value). The terms encompass elastic/elastomeric materials as well as materials that stretch but do not significantly retract (that is do not significantly recover upon being stretched and released) such as, for example, necked nonwoven materials and inherently extensible nonwoven materials like bonded carded webs. While many of the embodiments described herein include stretchable components, it is also desirable in alternative embodiments that such stretchable components be elastic and elastomeric as well.

“Elastic” or “Elasticized” means that property of a material or composite by virtue of which it tends to recover some size and shape after removal of a force causing a deformation.

The term “in liquid communication” refers to two or more adjacent materials or structures where moisture, urine, body exudates and the like are able to transfer from one to another when the product is worn as intended. The force driving this transfer can be capillary attraction, gravity, pressure generated by body movements or the like.

The term “insult” refers both to the natural deposition of a body exudate, and in particular urine or menses liquids during absorbent product use by a consumer, as well as the deposition of simulated body exudates during absorbent product testing.

As used herein, the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited onto a collecting surface. The foregoing patent is hereby incorporated by reference in its entirety.

As used herein, the term “machine direction” or MD means the length of a fabric in the direction in which it is produced. The term “cross direction” or “cross machine direction” or CD means the width of fabric, i.e. a direction generally perpendicular to the MD.

“Member” when used in the singular can refer to a single element or a plurality of elements.

As used herein, the term “necked material” refers to any material which has been constricted in at least one dimension by processes such as for example, drawing or gathering.

As used herein, the term “neckable material” refers to any material which can be necked. See for instance U.S. Pat. No. 4,965,122 which is incorporated in its entirety by reference hereto.

“Neck-bonded” laminate refers to a composite material having a neckable member that is bonded to a non-necked member (such as an elastic member) while the neckable member is extended in the machine direction creating a necked material that is elastic in the cross-direction. Examples of neck-bonded laminates are disclosed in U.S. Pat. Nos. 4,965,122; 4,981,747; 5,226,992; and 5,336,545, which are incorporated herein by reference in their entirety for all purposes.

As used herein the term “nonwoven fabric or web” means a web having a structure of individual fibers or threads which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

As used herein the term “polymer” generally includes but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the molecule. These configurations include, but are not limited to isotactic, syndiotactic and random symmetries.

As used herein, the term “spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns. The fibers may also have shapes such as those described in U.S. Pat. No. 5,277,976 to Hogle et al., U.S. Pat. No. 5,466,410 to Hills and 5,069,970 and 5,057,368 to Largman et al., which describe fibers with unconventional shapes. Each of the foregoing patents are hereby incorporated by reference in their entirety.

“Stretch-bonded” laminate refers to a composite material having at least two layers in which one layer is a gatherable layer and the other layer is an elastic layer. The layers are joined together when the elastic layer is in an extended condition so that upon relaxing the layers, the gatherable layer is gathered. For example, one elastic member can be bonded to another member while the elastic member is extended at least about 25% of its relaxed length. Such a multilayer composite elastic material may be stretched until the non-elastic layer is fully extended. Examples of stretch-bonded laminates are disclosed, for example, in U.S. Pat. Nos. 4,720,415, 4,789,699, 4781,966, 4,657,802, 4,655,760, 5,385,775 and U.S. patent application Ser. No. US2002-0104608 which are incorporated herein by reference in their entirety for all purposes.

As used herein, the term “superabsorbent material” refers to a water swellable, water insoluble organic or inorganic material capable, under the most favorable conditions, of absorbing more than 10 times its weight in an aqueous solution containing 0.9 weight percent sodium chloride, and desirably between about 10 to 30 or more times its weight. Organic materials suitable for use as a superabsorbent material of the present invention may include natural materials such as agar, pectin, guar gum, and the like; as well as synthetic materials, such as synthetic hydrogel polymers. Such hydrogel polymers include, but are not limited to, alkali metal salts of polyacrylic acids, polyacrylamides, polyvinyl alcohol, ethylene maleic anhydride copolymers, polyvinylethers, hydroxypropylcellulose, polyvinymorpholinone, polyvinylpyrrolidone; and polymers and copolymers of vinylsulfonic acid, polyacrylates, polyacrylamides, polyvinylpyridine, and the like. Other suitable polymers include hydrolyzed acrylonitrile grafted starch, acrylic acid grafted starch, and isobutylene maleic anhydride copolymers and mixtures thereof. It should be recognized that copolymers of combinations of the above materials and physical mixtures of combinations of the above materials may also be included. These hydrogel polymers are desirably lightly crosslinked to render the material substantially water insoluble. Crosslinking may, for example, be by covalent, ionic, van der Waals, or hydrogen bonding. The superabsorbent materials may be in any form suitable for use in absorbent composites including particles, fibers, flakes, spheres, films, and the like.

As used herein the term “thermal point bonding” refers to passing a fabric or web of fibers to be bonded between a heated calender roll and an anvil roll. The calender roll is usually, though not always, patterned in some way so that the entire fabric is not bonded across its entire surface, and the anvil roll is usually smooth. As a result, various patterns for calender rolls have been developed for functional as well as aesthetic reasons. One example of a pattern has points and is the Hansen Pennings or “H&P” pattern with about a 30% bond area with about 200 bonds/square inch as taught in U.S. Pat. No. 3,855,046 to Hansen and Pennings, incorporated herein by reference in its entirety. The H&P pattern has square point or pin bonding areas wherein each pin has a side dimension of 0.038 inches (0.965 mm), a spacing of 0.070 inches (1.778 mm) between pins, and a depth of bonding of 0.023 inches (0.584 mm). The resulting pattern has a bonded area of about 29.5%. Another typical point bonding pattern is the expanded Hansen Pennings or “EHP” bond pattern which produces a 15% bond area with a square pin having a side dimension of 0.037 inches (0.94 mm), a pin spacing of 0.097 inches (2.464 mm) and a depth of 0.039 inches (0.991 mm). Another typical point bonding pattern designated “714” has square pin bonding areas wherein each pin has a side dimension of 0.023 inches, a spacing of 0.062 inches (1.575 mm) between pins, and a depth of bonding of 0.033 inches (0.838 mm). The resulting pattern has a bonded area of about 15%. Yet another common pattern is the C-Star pattern which has a bond area of about 16.9%. The C-Star pattern has a cross-directional bar or “corduroy” design interrupted by shooting stars. Other common patterns include a diamond pattern with repeating and slightly offset diamonds with about a 16% bond area and a wire weave pattern looking as the name suggests, e.g. like a window screen, with about a 19% bond area. Typically, the percent bonding area varies from around 10% to around 30% of the area of the fabric laminate web. As is well known in the art, the spot bonding holds the laminate layers together as well as imparts integrity to each individual layer by bonding filaments and/or fibers within each layer.

As used herein, the term “ultrasonic bonding” means a process performed, for example, by passing the fabric between a sonic horn and anvil roll as illustrated in U.S. Pat. No. 4,374,888 to Bornslaeger, incorporated by reference herein in its entirety.

Various aspects and embodiments of the invention will be described in the context of a disposable absorbent article garment, such as a child's training pant. It is, however, readily apparent that the present invention could also be employed with other articles, disposable diapers, feminine care articles (for instance pantiliners and pads), adult incontinence garments and the like. Typically, the disposable articles are intended for limited use and are not intended to be laundered or otherwise cleaned for reuse. A child's training pant, for example, is discarded after it has become soiled by the wearer. In its various aspects, the invention can provide a distinctive absorbent article which exhibits desirable physical properties, such as softness, flexibility, conformance, trim appearance, reduced gapping and reduced leakage, particularly from around the sides of the article. The article can include an absorbent body structure which exhibits desirable physical properties, such as improved softness, flexibility, durability, conformance and stretchability. As a result, the absorbent structures and articles of the invention can provide increased strength, improved fit, reduced leakage, and reduced clumping, bunching or sagging during use.

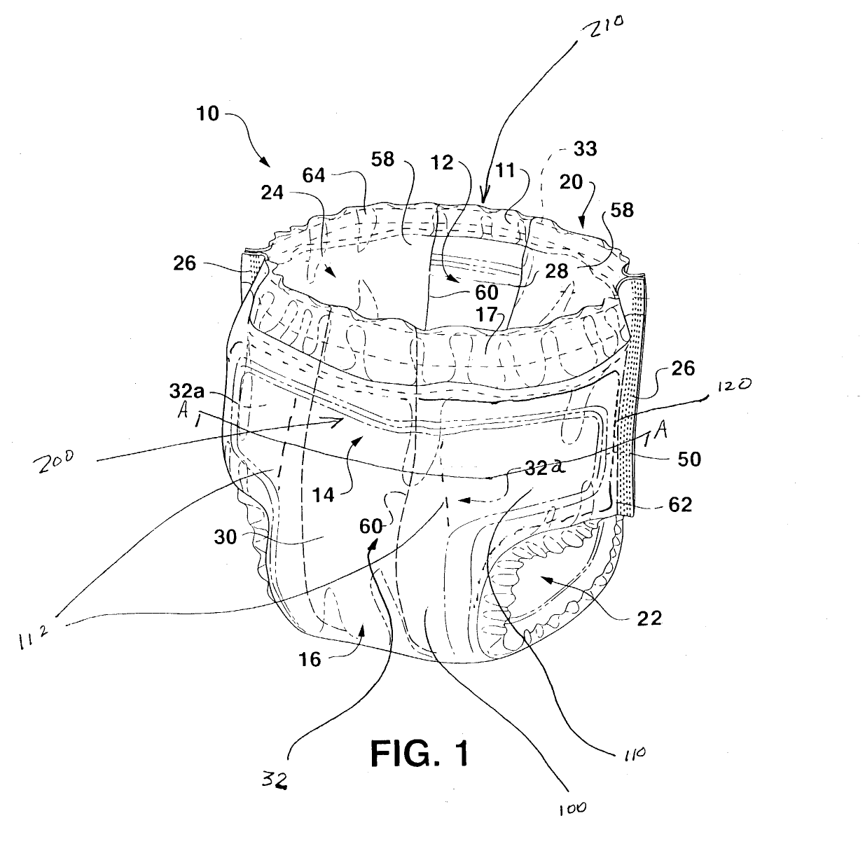

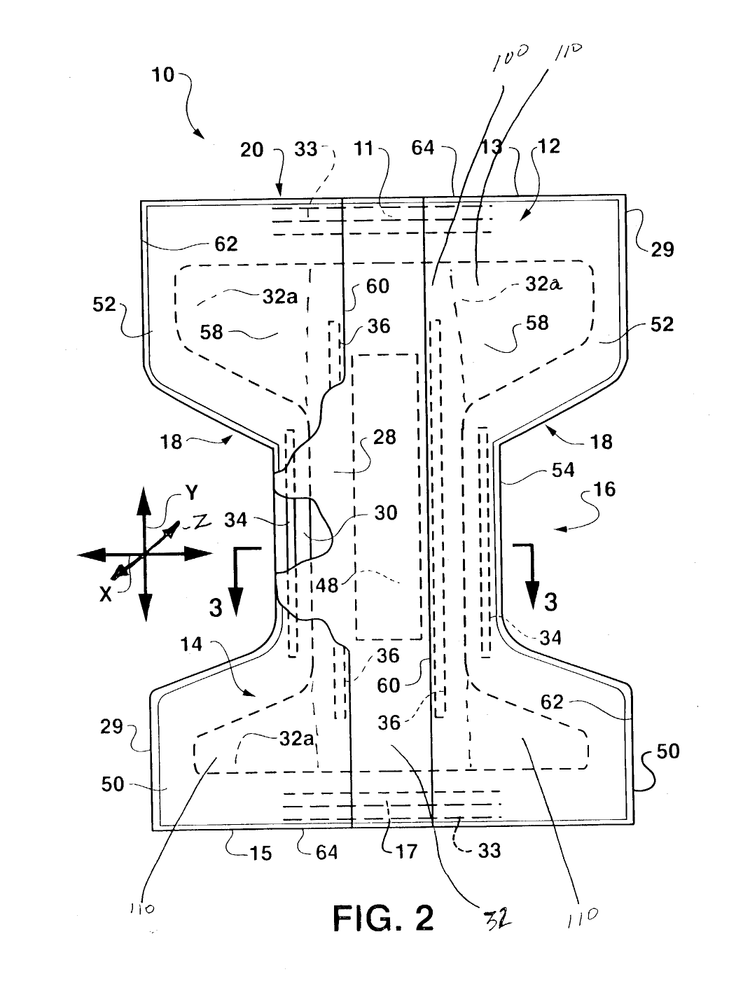

With reference to the figures, an article, such as that representatively shown as a child's training pant 10 in FIG. 1, includes a body or chassis 20 having a lengthwise, longitudinal direction Y (FIG. 2), a lateral, transverse cross-direction X, a Z-direction, a front waist region 14 on a front side 200, a back waist region 12 on a backside 210, and an intermediate crotch region 16 interconnecting the front and back waist regions. The waist regions 12 and 14 comprise those portions of the article 10 which when worn, wholly or partially cover or encircle the waist or mid-lower torso of the wearer. In particular configurations, the front 14 and back 12 waist regions may include elastic front and back waistband portions 17, 11. In the illustrated embodiments, the elastic waistband portions 11, 17 are generally continuous around the waist opening 24 of the article. In an alternate embodiment, the waistband portions 11, 17 may extend only partially across their respective waist regions. The intermediate crotch region 16 lies between and interconnects the waist regions 14 and 12, and comprises that portion of the article 10 which, when worn, is positioned between the legs of the wearer and covers the lower torso of the wearer. Thus, the intermediate crotch region 16 is an area where repeated liquid and/or waste insults typically occur in the training pant or other disposable absorbent article when the wearer is in an upright position.

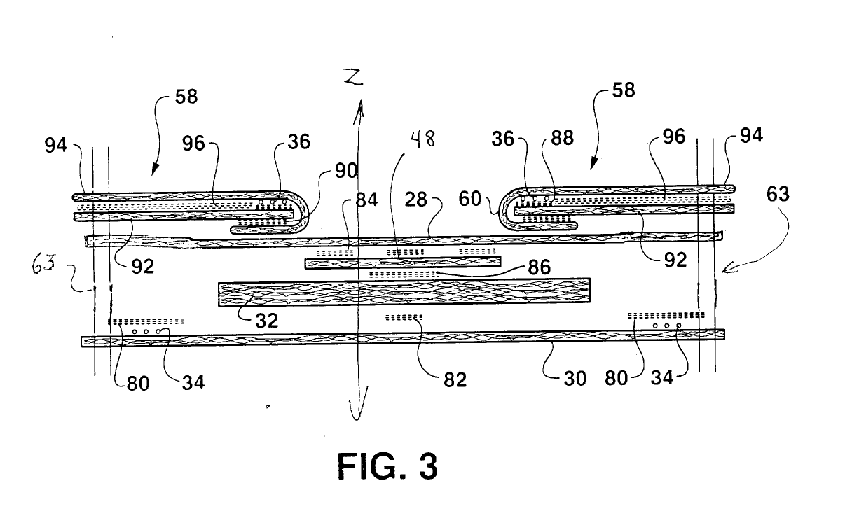

The article 10, as shown in FIG. 3, includes a substantially liquid-impermeable outer cover member 30, a liquid-permeable bodyside liner 28, and an absorbent body structure 32 sandwiched between the outer cover member 30 and the bodyside liner layer 28. The absorbent body structure (also referred to as an absorbent layer or retention layer) may be secured to the outer cover member 30 by an adhesive 82, and secured to the bodyside liner 28 by an adhesive 84, as represented in FIG. 3. The absorbent body structure may include laterally extending wing portions 32a, which extend along the sides of the garment along laterally extending ear portions 50, 52 of the chassis. Such laterally extending wing portions may be contiguous with a central absorbent portion 100 as seen in FIGS. 1 and 2, or may be separate from a central absorbent portion, shown as 110, but in close proximity with said central body absorbent structure portion, such that they are situated immediately adjacent the central portion and are in liquid communication with the central portion should liquid be present in the central portion. Such close proximity is indicated by broken lines 112 in FIG. 1 indicating that such wing portions may be separate but adjacent to the central absorbent portion. Such laterally extending wing absorbent portions 32a may extend up to, and in some circumstances into the side seam bond 26, as can be seen by the progressive phantom/broken line 120 approaching the side seam 26.

In certain embodiments, a surge management layer 48 may be optionally located adjacent the absorbent structure and attached by way of adhesive 86 (FIGS. 2 and 3). A surge management layer 48 helps to decelerate and diffuse surges or gushes of liquid that may be rapidly introduced into the absorbent body of the article. Desirably, the surge management layer can rapidly accept and temporarily hold the liquid prior to releasing the liquid into the storage or retention portions of the absorbent structure. The surge layer can be located below the bodyside liner layer 28. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference in their entirety for all purposes.

In the illustrated embodiment of the representative article 10, as shown in FIG. 2, the chassis 20 includes laterally extending front ear portions 50 and back ear portions 52. In the illustrated embodiment, the ear portions are an extension of the chassis structure and may be, for example, extensions of the outer cover member 30, bodyside liner 28, absorbent body structure, or a combination of such. This configuration is desirable particularly if the chassis 20, is stretchable in the transverse direction X, particularly across the front and back waist regions 14, 12. For example, such a chassis may include an elastomeric cover member 30, an elastomeric bodyside liner 28, and any combination of other elastomeric components that in combination render a stretchable unitary chassis.

As previously indicated, the absorbent body structure 32 includes laterally extending wing portions 32a, as seen in phantom lines in FIGS. 1 and 2. The wing portions extend at least partially along the laterally extending chassis ear portions 50, 52. Also, as previously stated, in a desirable embodiment, the wing portions 32a extend to a location immediately adjacent the side seams 26 and thus the absorbent body structure 32 generally encircles the waist region of a wearer, when the article is being worn.

The training pant 10 may be of a style and configuration wherein the front and back ear portions 50, 52 have lateral sides 29 that are brought together upon folding the chassis to form a pant structure having the waist opening 24 and leg openings 22. The lateral sides 29 cooperate with the lateral sides of the crotch region 16 of the chassis 20 to define curved leg contours 18, as particularly seen in FIG. 2. The curved leg contours 18 define the leg openings 22 when the article is assembled. The lateral sides 29 are bonded in a known manner so as to define side seams 26 of the pant structure. With this type of configuration, the pant 10 is pulled on by the wearer in a manner similar to underwear. Desirably, in one embodiment these seams 26 may be separable or tearable so that the pant 10 may be removed from the wearer by tearing the seams 26 and removing the article in a manner similar to a diaper. In an alternate embodiment, the front and back ear portions 50, 52 may be separable and re-attachable (refastenable) from one another at the side seams 26. A fastening system, such as a hook-and-loop system, or a refastenable tape system, may be used to interconnect the first waist region 12 with the second waist region 14 to define the pant structure and hold the article on a wearer. Additional suitable releasable fastening systems are described in U.S. Pat. No. 6,231,557 B1 and the International Application WO 00/35395, these references being incorporated herein by reference in their entirety for all purposes.

The article 10 also may incorporate longitudinally extending leak guards 58 (also known as “containment flaps” in the art) disposed over the bodyside liner 28. The guards 58 have longitudinal ends 64 that are attached to the chassis 20 generally at longitudinal ends 13, 15 thereof. For example, the guards 58 may extend over and be attached to the waist band portions 11, 17, for example by ultrasonic bonding. The guards 58 have an outboard lateral side 62 that is attached to the chassis desirably, but not necessarily outboard of the underlying absorbent body structure 32. The outboard sides 62 may in particular embodiments have a shape and contour corresponding generally to that of the front and back ear portions 50, 52 such that the guards 58 extend laterally beyond the wing portions 32a of the absorbent body structure. For example, the laterally outboard sides 62 may be generally coextensive with the lateral sides 29 of the chassis 20 and be bonded with the side seams 26. Referring to FIG. 3, the lateral outboard sides 62 may be attached along the crotch region 16 and contoured sides of the ear portions 50, 52 by way of, for example, a generally continuous ultrasonic bond line 63. A bead or spray of adhesive may also be used in place of or in conjunction with an ultrasonic bond. In addition a pressure bond made by fusing the layers together under high pressure may also be use in place of, or in combination with adhesives or sonic bonds. The guards 58 have a laterally inboard “free” side 60 that extends above and over the underlying absorbent body structure 32 such that the guards essentially define a containment pocket along the lateral sides of the absorbent structure 32.

A wide range of materials are suitable for use as the leak guards 58. For example, the guards 58 may be a nonwoven material such as a spunbond, meltblown, spunlaced, or carded polymeric material, a film material such as a polyolefin or polyurethane film, a foam material, or combinations thereof. The guards may also include materials described herein as being suitable for the outer cover member 30 or liner 28. The guards 58 may be substantially liquid impermeable, may be permeable to only gas, or may be permeable to both gas and liquid. It is generally desired that the guards 58 be formed from a material which is substantially impermeable to liquids, and which provides a cloth-like feel to the wearer. For example, the guards may contain a non-woven/liquid impervious film laminate construction. The guards may be formed of a woven or non-woven web which has been totally or partially treated to impart a desired level of liquid impermeability, or wettability and hydrophilicity.

In one embodiment, it is also generally desirable that the outer or body facing surface of the guards 58 be at least partially wettable or hydrophilic to at least partially absorb/retain any body exudates which become trapped between the guards and the wearer. In embodiments wherein the guards 58 are formed of a generally non-elastomeric material, provision can if desired be made to ensure that the guards are stretchable with the portions of the chassis 20 to which they are attached. For example, the longitudinal ends of the guards 58 may be gathered where attached to the elastic waistband portions 11, 17 so as to stretch therewith.

As discussed, the leak guards 58 may desirably be formed substantially of an elastomeric material or elastomeric composite material so as to be stretchable in the transverse direction. In this manner, regardless of the points of attachment of the guards laterally or at their respective longitudinal ends, the guards will not detract from the desired stretchable characteristics of another component of the article. The leak guards 58 are not limited to any particular type of elastomeric material, but in one embodiment, are desirably moisture resistant and desirably present a cloth-like feel to the wearer. Suitable materials may be, for example, a neck-bonded laminate, stretch-bonded laminate, stretchthermal laminate, or any combination of materials that provides moisture resistance and a desired degree of cloth-like feel to the wearer. A particularly suitable material is a neckbonded laminate (NBL) of an elastic polyethylene film and neck stretched spunbond polypropylene. As represented in FIG. 3, the guards 58, may be a laminate composite of a liquid impervious elastic film 92 and the necked spunbond web 94. An adhesive 96 is used to laminate the materials together; alternatively or in addition, ultrasonic bonds/welding can be used to laminate the materials. Elastic strands 36 may be incorporated into the laminating process with an adhesive 88 to produce the type of structure shown in FIG. 3. An extension or “flap” (not separately illustrated) of the necked spunbond web 94 may be folded over along the free side 60 of the guards 58 and attached to the underside of the elastic film 92 with an adhesive 90. Suitable other constructions and arrangements for the leak guards 58 are generally known to those skilled in the art and examples are also described in U.S. Pat. No. 4,704,116, incorporated by reference herein in its entirety for all purposes.

FIG. 2 shows a plan view of the representative training pant 10 in its generally flatout, (i.e., with substantially all elastic induced gathering and contraction removed). In this view, the bodyside surface of the pant 10 which contacts the wearer is facing the viewer, and portions of the structure are partially cut away to more clearly show the interior construction of the article. The outer edges of the article 10 define a periphery with longitudinally extending lateral sides and laterally extending ends 13, 15. The longitudinal ends 13, 15 are shown as straight, but optionally, may be curvilinear. The lateral sides are defined by the lateral most sides 29 of the front and back ear portions 50, 52, and lateral sides 54 of the crotch region 16. Laterally inwardly extending curved leg contours 18 are defined generally between the front and back ear portions and extend longitudinally generally along the crotch region 16. As mentioned, when the pant structure is formed by joining the lateral sides 29 of the front and back ear portions 50, 52, these contours 18 define leg openings for the article 10.

Elastomeric gathering members, such as leg elastics 34 and waist elastics 33 may be provided, as is well known in the art. The liner 28, outer cover 30, absorbent structure 32, surge layer 48, and elastic members 34 and 36 may be assembled together into a variety of well-known absorbent article configurations.

The leg elastic members 34 may be located in the lateral side margins of the chassis 20, particularly along the lateral sides of the crotch region 16, and are configured to draw and hold the chassis 20 against the legs of the wearer. The elastic members 34 are secured to the chassis 20, for example with an adhesive 80, in an elastically contracted state so that in a normal under-strain condition, the elastic members 34 effectively contract against the chassis. The use of elastic leg members in absorbent articles such as disposable diapers and training pants is widely known and understood in 20 the art.

The use of elastic waistbands is also widely known and used in the art. In the illustrated embodiment of FIG. 1, the waist elastics 33 are provided generally across the entirety of the front and back waistbands 11, 17. In alternate embodiments, the waist elastics 33 may extend only partially across the front and back waistbands, as represented in FIG. 2. The waist elastics 33 may be composed of any suitable elastomeric material, such as an elastomeric film, an elastic foam, multiple elastic strands, an elastomeric fabric, and the like. Embodiments of waistband structures that may be utilized with articles according to the invention are also described in U.S. Pat. Nos. 5,601,547; 6,358,350 B1; 6,336,921 B1; and 5,711,832, incorporated by reference herein in their entirety for all purposes.

The liner 28 and outer cover member 30 may be generally coextensive, and may have length and width dimensions which are generally larger than and extend beyond the corresponding dimensions of the absorbent structure 32. The extending portions define the corresponding side and end margins that allow for the waistbands 11, 17, leg elastics 34, and side seams 26 used, to be attached together with the outer cover member 30, liner 28, and leak guards 58. Optionally, the bodyside liner 28 and outer cover member 30 may not be coextensive. The outer cover member 30 may be composed of a liquid permeable material, but desirably comprises a material which is configured to be substantially impermeable to liquids. For example, a typical outer cover can be manufactured from a thin plastic film, a composite laminate, or other flexible, substantially liquid-impermeable material. As used in the present specification, the term “flexible” refers to materials which are compliant and which will readily conform to the general shape and contours of the wearer's body.

Alternative constructions of the outer cover member 30 may comprise a woven or non-woven fibrous web layer which has been totally or partially constructed or treated to impart the desired levels of liquid impermeability to selected regions that are adjacent or proximate the absorbent body structure. For example, the outer cover may include a gas-permeable, nonwoven fabric layer laminated to a polymer film layer which may or may not be gas-permeable. Other examples of fibrous, cloth-like outer cover materials can comprise a stretch thinned or stretch thermal laminate material. Although the outer cover 30 typically provides the outermost layer of the article, optionally the article may include a separate outer cover component member which is additional to the outer cover member.

In a particular embodiment, the outer cover 30 may be formed substantially from an elastomeric material and is thus stretchable. In this embodiment, the outer cover 30 may define the entire front and back waist regions 14, 12, including the front and back ear portions 50, 52. The outer cover 30 may, for example, be composed of a single layer, multiple layers, laminates, spunbond fabrics, films, meltblown fabrics, elastic netting, a microporous web, bonded carded webs or foams comprised of elastomeric or polymeric materials. Elastomeric nonwoven laminate webs may include a nonwoven material joined to one or more gatherable nonwoven webs, films, or foams. Stretch Bonded Laminates (SBL) and Neck Bonded Laminates (NBL) are examples of elastomeric composites. Examples of suitable materials are Spunbond-Meltblown fabrics, Spunbond-Meltblown-Spunbond fabrics, Spunbond fabrics, or laminates of such fabrics with films, foams, continuous filament strands or other nonwoven webs. Elastomeric materials may include cast or blown films, foams, or meltblown fabrics composed of polyethylene, polypropylene, or polyolefin copolymers, as well as combinations thereof. The elastomeric materials may include PEBAX elastomer (available from AtoChem located in Philadelphia, Pa.), HYTREL elastomeric polyester (available from E. I. DuPont de Nemours located in Wilmington, Del.), KRATON elastomer (available from Kraton Company located in Houston, Tex.), or strands of LYCRA elastomer (available from E. I. DuPont de Nemours located in Wilmington, Del.), or the like, as well as combinations thereof. The outer cover 30 may include materials that have elastomeric properties through a mechanical process, printing process, heating process, or chemical treatment. For examples such materials may be apertured, creped, neck-stretched, heat activated, embossed, and micro-strained; and may be in the form of films, webs, and laminates.

The bodyside liner 28 presents a body-facing surface which is compliant, soft-feeling, and non-irritating to the wearer's skin. Further, the bodyside liner 28 can be less hydrophilic than the absorbent body 32, and is sufficiently porous to be liquid permeable, permitting liquid to readily penetrate through its thickness to reach the absorbent body structure. A suitable bodyside liner layer 28 may be manufactured from a wide selection of web materials, such as porous foams, reticulated foams, apertured plastic films, natural fibers (for example, wood pulp or cotton fibers), synthetic fibers (for example, polyester or polypropylene fibers), or a combination of natural and synthetic fibers. The bodyside liner layer 28 is typically employed to help isolate the wearer's skin from liquids held in absorbent body 32.

Various woven and nonwoven fabrics, and apertured films, and/or foams can be used for the bodyside liner 28. For example, the bodyside liner may include a meltblown web, a spunbonded web, or a bonded-carded-web composed of the desired fibers. The various fabrics can be composed of natural fibers, synthetic fibers or combinations thereof.

In particular aspects, the bodyside liner 28 may be comprised of polymer fibers, networks, laminates, liquid permeable films, cellulosic fibers, rayon, water swellable gels, and elastomeric materials, as well as combinations thereof. Suitable materials for the bodyside liner can include meltblown webs, airlaid webs, spunbond webs, or bonded-carded webs of synthetic continuous or discrete polymer fibers and/or natural fibers, a pattern bonded spunbonded web, airlaid web, or bonded carded web, as well as combinations thereof. Suitable polymers can include polypropylene, polyethylene, polyester, and bicomponent materials composed of these polyolefins.

The bodyside liner fabrics may be composed of a substantially hydrophobic material, and the hydrophobic material may optionally be treated with a surfactant or otherwise processed to impart a desired level of wettability and hydrophilicity. In a particular embodiment of the invention, the bodyside liner 28 can be a nonwoven, spunbond polypropylene fabric which is necked approximately 60%. Strands of KRATON G2760 compound elastomer material may be adhered to the necked spunbond material.

The fabric can be surface treated with an operative amount of surfactant, such as about 0.45% AHCOVEL Base N62 surfactant, available from Uniqema, a division of ICI, a business having offices located in New Castle, Del. The surfactant can be applied by any conventional means, such as spraying, printing, brush coating or the like.

In particular embodiments wherein it is desired that the bodyside liner layer 28 be stretchable, suitable elastomeric materials can include elastic strands, LYCRA® elastics, elastic films, cast or blown; nonwoven elastic webs, meltblown or spunbond elastomeric fibrous webs, as well as combinations thereof. Additionally, such stretchable liners can include stretchable bicomponent fibrous spun materials. Such bicomponent spun materials may be, for example, sheath/core nonwoven fibrous fabric. The core of such fibers may be a styrenic block copolymer, such as a Kraton elastomer, and the sheath may be a single-site catalyzed polyolefin such as metallocene catalyzed polyethylene, or other polyolefin, such as polypropylene. Examples of elastomeric materials include KRATON elastomers, HYTREL elastomers, ESTANE elastomeric polyurethanes (available from B.F. Goodrich and Company located in Cleveland, Ohio), or PEBAX elastomers. The bodyside liner may include blends or laminates of fibers, scrim, webs, and films with perforations, apertures, creping, heat activation, embossing, micro-straining, chemical treatment, or the like, as well as combinations thereof.

The bodyside liner 28 and outer cover 30 are connected or otherwise associated together in an operable manner. As used herein, the term “associated” encompasses configurations in which the bodyside liner 28 is directly joined to the outer cover 30 by affixing the bodyside liner 28 directly to the outer cover 30, and configurations wherein the bodyside liner 28 is indirectly joined to the outer cover 30 by affixing the bodyside liner 28 to intermediate members which in turn are affixed to the outer cover 30. The bodyside liner 28 and outer cover 30 can, for example, be joined to each other in at least a portion of the training pant periphery by suitable attachment mechanisms (not shown) such as adhesive bonds, ultrasonic (sonic) bonds, thermal bonds, pinning, stitching or any other attachment technique known in the art, as well as combinations thereof. For example, a uniform continuous layer of adhesive, a patterned layer of adhesive, a sprayed pattern of adhesive or an array of separate lines, swirls or spots of construction adhesive may be used to affix the bodyside liner 28 to the outer cover 30. It should be readily appreciated that the above-described attachment means may also be employed to suitably interconnect, assemble and/or affix together the various other component parts of the articles which are described herein.

The absorbent body (material) structure 32 (including wing portions 32a and optional surge layer 48) is positioned between the outer cover 30 and the bodyside liner 28. The absorbent body structure 32 can be any structure or combination of components which are generally compressible, conformable, non-irritating to a wearer's skin, and capable of absorbing and retaining liquids and certain body wastes. For example, the structure 32 may include an absorbent web material of cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular embodiment, the absorbent web material is a matrix of cellulosic fluff and superabsorbent hydrogel-forming particles. The cellulosic fluff may comprise a blend of wood pulp fluff. One preferred type of fluff is identified with the trade designation CR 1654, available from U.S. Alliance of Childersburg, Ala., USA, and is a bleached, highly absorbent sulfate wood pulp containing primarily soft wood fibers. The absorbent materials may be formed into a web structure by employing various conventional methods and techniques. For example, the absorbent web may be formed with a dry-forming technique, an air forming technique, a wet-forming technique, a foam-forming technique, or the like, as well as combinations thereof. Methods and apparatus for carrying out such techniques are well known in the art. Furthermore, the absorbent body structure may itself encompass multiple layers in the Z direction. Such multiple layers may take advantage of differences in absorbency capacity, such as by placing a lower capacity absorbent material layer closer to the liner 28 and a higher capacity absorbent material closer to the outercover layer 30. Likewise, discrete portions of an absorbent body single-layered structure may encompass higher capacity absorbents, and other discrete portions of the structure may encompass lower capacity absorbents.

As a general rule, the superabsorbent material is present in the absorbent web in an amount of from about 0 to about 90 weight percent based on total weight of the web. The web may have a density within the range of about 0.10 to about 0.35 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 10 times its weight in liquid, and desirably is capable of absorbing more than about 25 times its weight in liquid. Suitable superabsorbent materials are readily available from various suppliers. For example, Favor 880, and Favor 9543 superabsorbents are available from Stockhausen GmbH of Germany; and Drytech 2035 is available from Dow Chemical Company, of Midland Mich., USA. Superabsorbent materials may be selected so as to strategically place higher capacity absorbent materials in one location or another within the absorbent body/material structure. For instance absorbent materials may be placed in the central portion, or in the laterally extending wing portions so that either portion demonstrates an absorbent capacity that is at least 10%, or greater, than the other portion.

After being formed or cut into a desired shape, the absorbent web material may be wrapped or encompassed by a suitable tissue wrap that aids in maintaining the integrity and shape of the absorbent structure 32.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers may be utilized as the melt-spun component of the coform material. For instance, in some embodiments, thermoplastic polymers can be utilized. Some examples of suitable thermoplastics that can be utilized include polyolefins, such as polyethylene, polypropylene, polybutylene and the like; polyamides; and polyesters. In one embodiment, the thermoplastic polymer is polypropylene. Some examples of such coform materials are disclosed in U.S. Pat. No. 4,100,324 to Anderson, et al.; 5,284,703 to Everhart, et al.; and 5,350,624 to Georger, et al.; which are incorporated herein in their entirety by reference thereto for all purposes.

In an absorbent article according to the present invention, at least a portion of the absorbent body structure 32 is elastomeric. For example, in the embodiment as shown in FIG. 1 the entire chassis 20 is desirably stretchable. The absorbent body structure 32 is stretchable so as not to inhibit the stretchability of other components.

For this purpose, the absorbent web material can include elastomeric fibers in an amount which is at least a minimum of about 2 wt %. The amount of elastomeric fibers can alternatively be at least about 3 wt %, and can optionally be at least about 5 wt % to provide improved performance. In addition, the amount of elastomeric fibers can be not more than about 60 wt %. Alternatively, the amount of elastomeric fibers can be not more than about 45 wt %, and optionally, can be not more than about 30 wt % to provide improved benefits. These values are important because they can provide the absorbent retention portion with desired levels of stretchability and structural stability without excessively degrading the physical properties or the liquid-management properties of the absorbent structure. An absorbent web material with an excessively low proportion of elastomeric fibers may be insufficiently stretchable, and a web material with an excessively high proportion of elastomeric fibers may exhibit an excessive degradation of its absorbency functionalities, such as poor intake, poor distribution, poor retention of liquid and/or an excessive tension force when stretched.

The absorbent body structure 32 may include an elastomeric coform absorbent web material. Such materials are described for instance in U.S. Pat. Nos. 6,231,557 B1 and 6,362,389 B1, which are each incorporated by reference herein in its entirety. In particular aspects, the elastomeric coform material can have an overall coform basis weight which is at least a minimum of about 50 g/m². The coform basis weight can alternatively be at least about 100 g/m² and can optionally be at least about 200 g/m² to provide improved performance. In addition, the coform basis weight can be not more than about 1200 g/m². Alternatively, the coform basis weight can be not more than about 900 g/m², and optionally, can be not more than about 800 g/m² to provide improved benefits. These values are important because they can provide the absorbent body structure with desired stretchability and structural stability without excessively degrading the physical properties or the liquid-management functionalities of the absorbent body structure. Retention portions having excessively low proportions of elastomeric coform material may not be sufficiently stretchable. An absorbent web material having excessively large amounts of elastomeric coform materials can exhibit an excessive degradation of their absorbency functionalities, such as an excessive degradation of intake, distribution and/or retention properties.

Other examples of usable elastomeric absorbent structures are described in U.S. Pat. Nos. 5,964,743, 5,645,542, 6,231,557, and 6,362,389 B1, incorporated by reference herein for all purposes.

The absorbent web material utilized in the absorbent body structure 32 is also selected so that the individual absorbent body structure possesses a particular individual total absorbency depending on the intended article of use. For example, for infant care products, the total absorbency can be within the range of about 300-900 grams of 0.9 wt % saline, and can typically be about 500 g of saline. For adult care products, the total absorbency can be within the range of about 1000-1600 grams of saline, and can typically be about 1300 g of saline. For feminine care products, the total absorbency can be within the range of about 7-50 grams of saline, and can typically be within the range of about 30-40 g of saline.

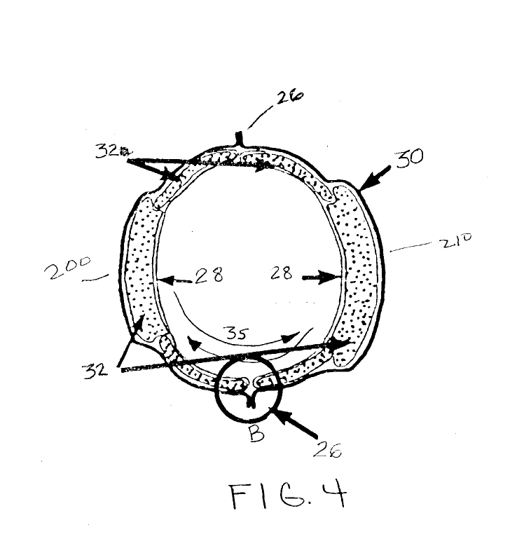

As can be seen in FIG. 4, which illustrates generally a cross-sectional view along line A-A of FIG. 1, of a chassis in accordance with one embodiment of the invention (however, for simplification, shown without a leak guard or surge layer). A stretch absorbent material of the absorbent body structure 32 wraps between the center of the training pant (throughout the crotch area 16) to the laterally extending side wing portions 32a, adjacent the side seam areas 26. The Figure shows the stretchable absorbent materials as separate but proximate components as in 100 and 110 of FIG. 1. Alternatively the absorbent could be one piece that extended all around the circumference of the garment. The stretch absorbent may include pulp fluff and/or superabsorbent material components and a stretch material as previously described. Such materials may be for example an elastomer coform composite of styrenic block copolymers and absorbents as previously described. In the front and back portions of the pant, for instance the central absorbent body portion 100, stretch may not be a necessity. Therefore, in these locations, non-stretch conventional pulp fluff/superabsorbent material (SAM) combinations may alternatively be utilized instead of stretchable absorbent materials.

In this embodiment, only a narrow gap separates the stretch absorbent materials adjacent the side seams 26, as the stretch absorbent material in the laterally extending side wing portions 32a are immediately adjacent one another as can be seen in the section labeled B of FIG. 4. If a user of such an article insults the pant in the lying/sleeping position, urine or waste will travel in approximately the paths identified by arrows 35 from the front/back of the pant along the side and around the user's hips, to the front/back absorbent areas. The waste can essentially flow from the point of insult around the hips to the reverse side, ideally in the case of urine, from front to back. This flow or movement of urine or waste can occur within and through the absorbent and over and under the absorbent or body side liner. Since these side stretch absorbent areas include stretch as well as absorbent materials, the pants provide both for a range of fit and ease of donning, while also including absorbent capacity where it is needed, either during sleep or in the reclining position.

In the stretch embodiment of FIG. 4, an extensible/stretchable liner 28 of the type previously described, is included, along with a stretchable outer cover 30 and the stretchable absorbent body structure 32. For instance, such a liner may be between about 0.25-0.3 osy polypropylene spunbond, that has been treated with a surfactant and necked to approximately 50 to 60 percent of its original width. Such a necked material would provide extension and in some circumstances retraction in the material cross-machine direction if part of a neck-bonded laminate including an elastomeric component..

The pant may be made with either a completely stretchable outer cover, as previously described, or alternatively, with a non stretchable outer cover, but with stretchable side panels as described in WO 0037009. In any event, the stretchable absorbent of laterally extending wing portions (of the absorbent body structure) is placed in the same areas of the stretchable side panels, such that the stretch of the side panel function is not hampered, but additional absorbent functionality is provided. Such stretchable absorbent material may be covered with a stretchable outercover and a stretchable liner as seen in the Figure.

The pant is also illustrated as including ultrasonically bonded side seams 26. It should be recognized however, that such side seams may instead be refastenable openings as previously described.

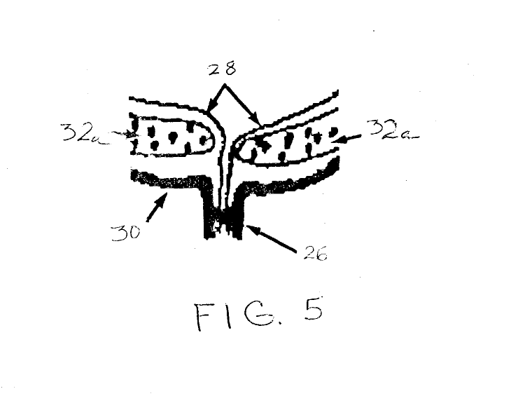

FIGS. 5, 6, and 12 each illustrate alternative means of forming the side seam in the ultrasonic side seam embodiment of the current invention. For instance, FIG. 5 is a close-up cross-sectional view of an alternative embodiment of the circled side seam area labeled B of FIG. 4. As can be seen in the Figure, the stretchable liner 28 passes between the adjacent end edges of the stretch absorbent laterally extending wing areas 32a, and between the stretchable outer cover 30 edges 29. The stretchable liner edges are held in place in the ultrasonic side seam 26.

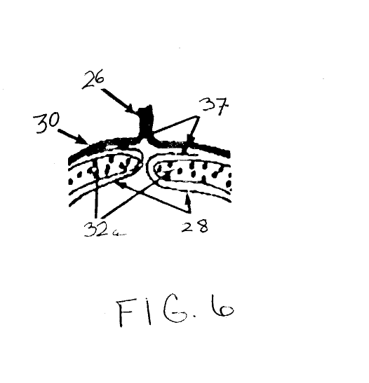

FIG. 6 is a close-up cross-sectional view of another alternative embodiment of the circled side seam area labeled B of FIG. 4. In the side seam area, the stretchable liner wraps around the edge of the stretch absorbent laterally extending wing materials 32a in the side seam location and is adhered in position with adhesive 37 to the inside surface of the stretchable outercover 30.

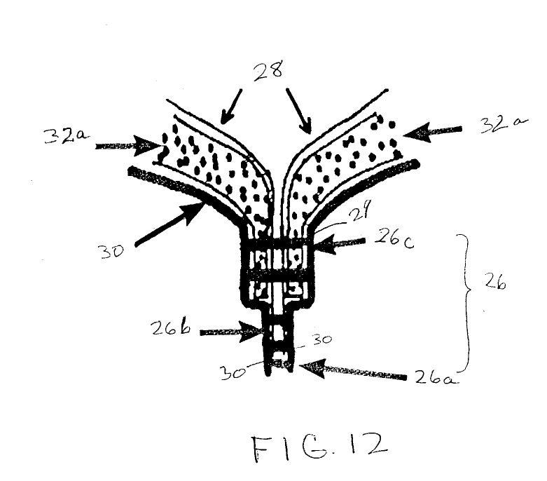

In a further alternative embodiment of the present invention, it may be desirable for the ultrasonic seam and side panel to be water impervious or waterproof. Since, any overflow of liquids from a user insult could flow by gravity across the short space between the front and back stretch absorbent materials in the laterally extending wing portions 32a, it would therefore be desirable to have a side seam area 26 which provides for extra protection from leakage. In this regard, FIG. 12 is a close-up cross-sectional view of a further alternative embodiment of the circled side seam area labeled B of FIG. 4. FIG. 12 illustrates stretchable liner 28 edges, laterally extending stretch absorbent wing portion 32a edges and stretchable outer cover 30 edges, maintained generally in an ultrasonic side seam 26. As can be seen in FIG. 12, only a portion of the ultrasonic bond is through the adjacent edges of the stretch absorbent material of the laterally extending wing portions 32a. This is accomplished if there is thermoplastic elastomeric material, such as the described styrenic block copolymer coform material, within the stretch absorbent material that is capable of holding an ultrasonic bond. The thermoplastic materials should flow at a temperature less than the ignition temperature of the remainder of the coform material for instance (i.e. pulp fluff). In such an embodiment, there is practically a continuous stretch absorbent material from the front of the pant to the back, through the ultrasonic seam area. Essentially, liquid can travel from one laterally extending wing portion of the absorbent body structure 32a across the stretchable liner 28 to the second laterally extending wing portion of the absorbent body structure. It is likely that such an arrangement would enhance the wicking of moisture around the hips of a wearer while in the sleeping or reclining position. As is also illustrated in the Figure, via bold dark lines (representing ultrasonic bond points) running perpendicular to the stretchable outercover edges 29, laterally extending wing portion edges and stretchable liner material edges, a portion of the ultrasonic bond 26 is a bond 26a between/through layers of the stretchable outer cover edges 30 only. A second portion of the ultrasonic bond 26b is between/through layers of the stretchable outer cover edges and the stretchable liner material edges. A third portion of the ultrasonic bond 26c is between/through all three, of the stretchable outercover edges, the stretchable liner edges and the stretchable absorbent edges. In this fashion, a portion of the ultrasonic bond not impacting the stretchable absorbent could then be used to seal/dam off the absorbent from the outside of the pant, (via the bond and the water impermeable outercover) thereby forming a moisture impervious bond for preventing leakage in this area of the article.

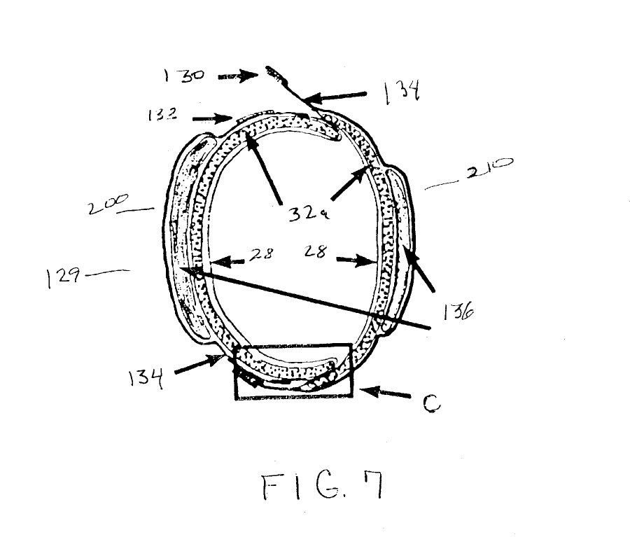

FIG. 7 is a cross-sectional view of an alternative embodiment of a pant chassis similar to that of FIG. 1, but including a refastenable opening along the hip areas, rather than an ultrasonically bonded side seam edge. In this embodiment, hook and loop fasters 130 and 132 provide the fastening system for the sides of the refastenable garment 129.

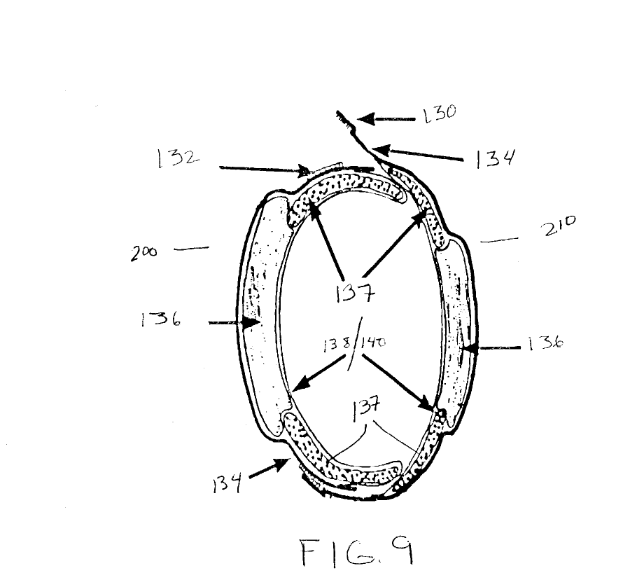

The garment includes a stretchable outer cover 30/refastenable stretchable side panel 134 on each side of the refastenable opening. Additionally, stretch absorbent materials are located in the laterally extending wing portions of the absorbent body structure 32a. The article also includes a stretchable liner 28 as in the previous embodiments. However, rather than have a single layer of absorbent or stretch absorbent, in an alternative embodiment, the absorbent material is present in two immediately adjacent layers, with a higher capacity absorbent material 136 positioned under lower capacity stretch absorbent material, that is closer to the stretchable outer cover. In this embodiment, the higher capacity absorbent occupies a layer in the central portion of the absorbent body structure, and the stretch absorbent occupies both a central portion of the absorbent body structure as well as laterally extending wing portions of the absorbent body structure. The stretchable absorbent is continuous throughout its layer, rather than being comprised of separate structures as in 100 and 110, or as seen in the embodiment of FIG. 9. The higher absorbent capacity material may itself demonstrate stretch functionality as well. In one particular embodiment, a higher stretch, lower absorbent material may be placed closer to the stretchable liner, and a higher capacity, lower stretch absorbent material may be placed closer to the stretchable outer cover. As can be seen from the figure, the laterally extending wing absorbent portions over-lap as the article is secured/fastened.

Essentially, the front and back absorbent areas over-lap to assist in preventing leakage through the refastenable openings, with one of the laterally extending wing portions of the absorbent body structure (and liner) overhanging the edge of the outercover/side panel 134.

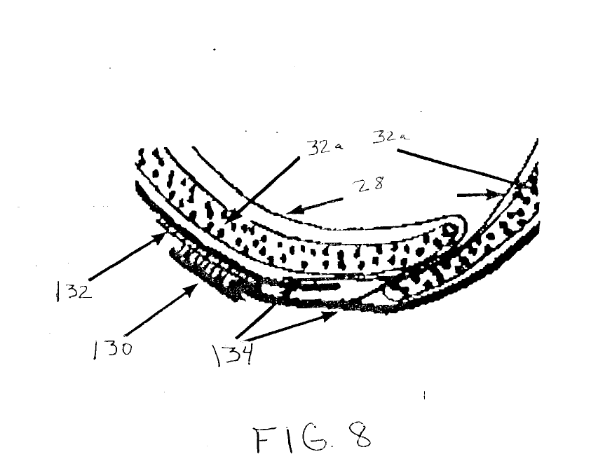

FIG. 8 illustrates a close-up cross-sectional view of the circled refastenable area labeled C of FIG. 7. As can be seen in the figure, the stretchable liner 28 is attached to the stretchable outer cover/side panels of the respective sides of the chassis. However, on one side of the chassis (the loop side), the liner wraps around the stretch absorbent material and both the liner and the stretch absorbent overhang the edge of the stretchable outer cover/side panel. This overhang allows for the necessary overlap of stretch absorbent laterally extending wing edges when the article is fastened together. In this embodiment, the loop component of the hook and loop fastening system is positioned on the chassis in a location away from the laterally extending ear edge, so as to allow for the overlap in the Z direction of the absorbent and liner materials. This overlap allows for the liquid communication necessary to move liquid from the front to the back of the article.

FIG. 9 illustrates a cross-sectional view of still a further alternative embodiment of a pant chassis similar to that of FIG. 1. However, as can be seen in this illustration, the pant chassis includes a high capacity absorbent material in a central portion, adjacent two separate laterally extending wing portions which contain the stretch absorbent material. The high capacity absorbent component 136 occupies a central position (as 100 in FIG. 1) in the chassis, and the laterally extending wing portions 137 wrap around the sides (as in 110 of FIG. 1). The stretch absorbent material of the laterally extending wing sides, is immediately adjacent (laterally) to the higher capacity absorbent material and is in liquid communication with it, by virtue of its close proximity to the higher capacity absorbent material 138, and its edges being partially engulfed or overlapped by the lateral edges of the higher capacity absorbent material 140 in the Z direction.

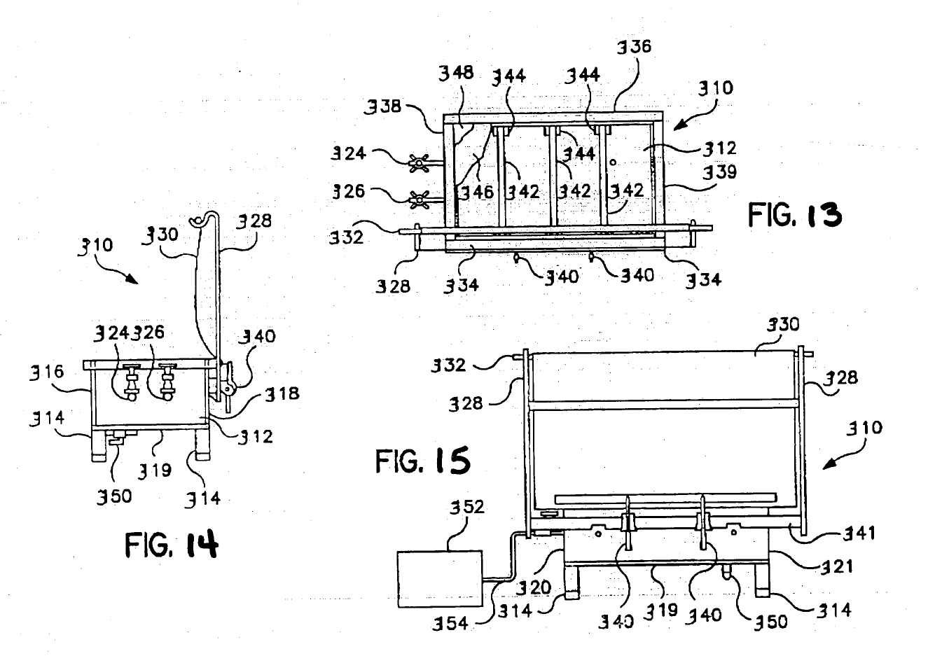

Saturated capacity can be determined using a Saturated Capacity (SAT CAP) Tester with Magnahelic vacuum gage and latex dam: Referring to FIGS. 13-15, a Saturated Capacity tester vacuum apparatus 310 comprises a vacuum chamber 312 supported on four leg members 314. Vacuum chamber 312 includes a front wall member 316, a rear wall member 318 and two side walls 320 and 321. The wall members are about 0.5 inch thick, and are constructed and arranged to provide a chamber having outside dimensions measuring 23.5 inches in length, 14 inches in width and 8 inches in depth. A vacuum pump (not shown) operably connects with vacuum chamber 312 through an appropriate vacuum line conduit and vacuum valve 324. In addition, a suitable air bleed line connects into vacuum chamber 312 through air bleed valve 326. A hanger assembly 328 is suitably mounted on rear wall 318 and is configured with S-curved ends to provide a convenient resting place for supporting latex dam sheet 330 in a convenient position away from the top of vacuum apparatus 310. A suitable hanger assembly can be constructed from 0.25 inch diameter stainless steel rod. Latex sheet 330 is looped around dowel member 332 to facilitate grasping and allow a convenient movement and positioning of the latex sheet. In the illustrated position, dowel member 332 is shown supported in hanger assembly 328 to position the latex sheet 330 in an open position away from the top of vacuum chamber 312. A bottom edge of latex sheet 330 is clamped against a rear edge support member 334 with suitable securing means, such as toggle clamps 340. The toggle clamps are mounted on rear wall member 318 with suitable spacers 341 which provide an appropriate orientation and alignment of the toggle clamps for the desired operation. Three support shafts 342 are 0.75 inch in diameter and are removably mounted within vacuum chamber 312 by means of support brackets 344. The support brackets are generally equally spaced along front wall member 316 and rear wall member 318 and arranged in cooperating pairs. In addition the support brackets are constructed and arranged to suitably position the uppermost portions of support shafts 342 flush with the top of the front, rear and side wall members of vacuum chamber 312. Thus, support shafts 342 are positioned substantially parallel with one another and are generally aligned with side wall members 320 and 321. In addition to rear edge support member 334, the tester apparatus includes a front support member 336 and two side support members 338 and 339. Each edge support member measures about 1 inch in width and about 1.25 inches in height. The lengths of the support members are constructed to suitably surround the periphery of the open top edges of vacuum chamber 312, and are positioned to protrude above the top edges of the chamber wall members by a distance of about 0.5 inch. A layer of egg crating type material 346 is positioned on top of support shafts 342 and the top edges of the wall members of vacuum chamber 312. The egg crate material extends over a generally rectangular area measuring 23.5 inches by 14 inches, and has a depth measurement of about 0.38 inches. The individual cells of the egg crating structure measure about 0.5 inch square, and the thin sheet material comprising the egg crating is composed of a suitable material, such as polystyrene. For example, the egg crating material can be McMaster Supply Catalog No. 162 4K 14, translucent diffuser panel material. A layer of 0.19 mesh nylon screening 348, which measures 23.5 inches by 14 inches, is placed on top of egg crating material 346. A suitable drain line and drain valve 350 connects to bottom plate member 319 of vacuum chamber 312 to provide a convenient mechanism for draining liquids from the vacuum chamber. The various wall members and support members of tester 310 may be composed of a suitable noncorroding, moisture-resistant material, such as polycarbonate plastic. The various assembly joints may be affixed by solvent welding, and the finished assembly of the tester is constructed to be watertight. A vacuum gauge 352 operably connects through a conduit into vacuum chamber 312. A suitable pressure gauge is a Magnahelic differential gauge capable of measuring a vacuum of 0-100 inches of water, such as a No. 2100 gauge available from Dwyer Instrument Incorporated.

In use, sample of absorbent structure is weighed and then placed in excess 0.9% saline solution and allowed to soak for 20 minutes. After the 20 minute soak time, the absorbent structure is placed on the egg crate material and mesh nylon screening of the Saturated Capacity Tester. The latex sheet is placed over the absorbent structure(s) and the entire egg crate grid so that the sheet creates a seal when vacuum is drawn on the Tester. A vacuum of 0.5 pounds per square inch (psi) is held in the Saturated Capacity Tester for five minutes. The vacuum creates a pressure on the absorbent structure(s), causing drainage of some liquid. After five minutes at 0.5 psi vacuum, the latex sheet is rolled back and the absorbent structure(s) are weighed to generate a wet weight. The overall capacity of each absorbent structure is determined by subtracting the dry weight of each absorbent from the wet weight of that absorbent determined at this point in the procedure. The 0.5 psi SAT CAP or SAT CAP of the absorbent structure is determined by the following formula: (wet weight—dry weight)/(dry weight). The SAT CAP value has units of grams liquid/gram absorbent. For both overall capacity and SAT CAP, a minimum of four specimens of each sample should be tested, and the results averaged. If the absorbent structure has low integrity or disintegrates during the soak or transfer procedures, the absorbent structure can be wrapped in a containment material such as paper toweling, for example HI-DRI® paper towels manufactured by Kimberly-Clark Corporation, Neenah, Wis. The absorbent structure can be tested with the overwrap in place, and the capacity of the overwrap can be independently determined and subtracted from the wet weight of the total wrapped absorbent structure to obtain a wet absorbent weight.

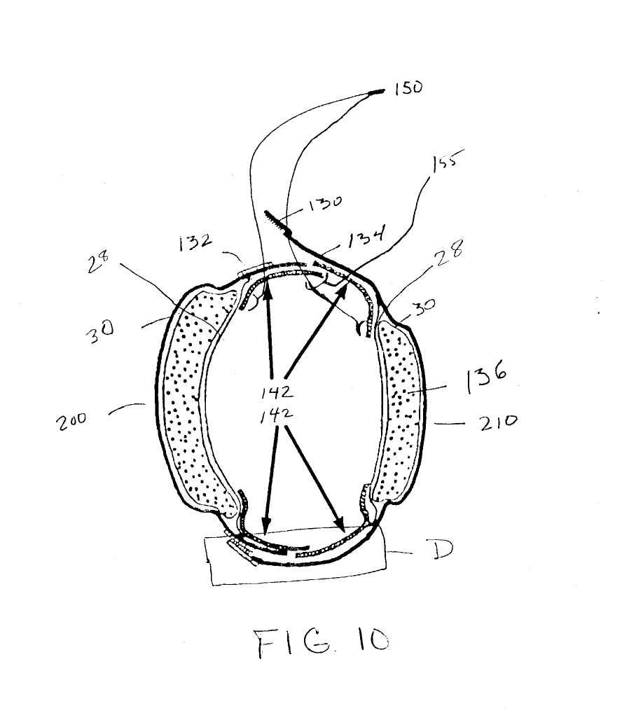

FIG. 10 likewise is a cross-sectional view of an alternative embodiment of a pant chassis similar to that of FIG. 9. However, rather than employing a distinctive laterally extending wing portion stretch absorbent material, a “bridge”-like absorbent material 142 may be employed. Such a “bridge”-like absorbent material 142 is a material designed to bridge one portion of a central portion of the absorbent body structure 136 to the other, such that liquid waste can flow from one side of the absorbent structure to the other while the wearer is in a reclining or sleeping position. Such “bridge”-like structure 142 may be comprised of just absorbent materials that have been fastened to the stretchable chassis in a somewhat gathered state such that they do not hinder the overall stretch functionality of the article, or alternatively, they may be stretchable and absorbent. Desirably, such materials do not retain large amounts of moisture, but rather act to transfer moisture to a portion of the absorbent body structure with excess unused capacity.

In embodiments wherein the “bridge”-like materials 142 are formed of a generally non-elastomeric material, provision is made to ensure that the “bridge”-like materials 142 are stretchable with the portions of the chassis 134 to which they are attached. For example, the “bridge”-like materials 142 may be gathered where attached to the stretchable liner 28 or outercover/side panel (30 and 134 respectively) so as to stretch therewith (to the extent they have exhausted their gathers).

As an example, as can be seen in FIG. 10, the “bridge”-like material is attached to both the stretchable liner 28 and the stretchable outer cover/side panels 134. The “bridge”-like materials (which are essentially separate laterally extending wing portions 32a of the absorbent body structure 32) overlap some of the central portion of the absorbent body structure in for instance, the Z direction (into the article). Additionally, as in previously described embodiments of the invention, the “bridge”-like materials are positioned along the chassis in such a fashion that they overlap each other 155, such as in the Z direction, when the article is worn and fastened shut by the wearer. In such a construction, liquid can easily travel from the central portion of the absorbent body structure, through the liquid permeable liner layer 28 and into the “bridge”-like materials, when the user is wearing the absorbent article in a reclining or sleeping position. From the “bridge”-like materials, the liquid can travel around the user's hip areas to the other side of the central portion of the absorbent body structure via the liner 28. It is likely that additional liquid storage capacity will be available on the other side of the absorbent structure, to accommodate excess moisture that may be generated while the user is sleeping or reclining on their side.

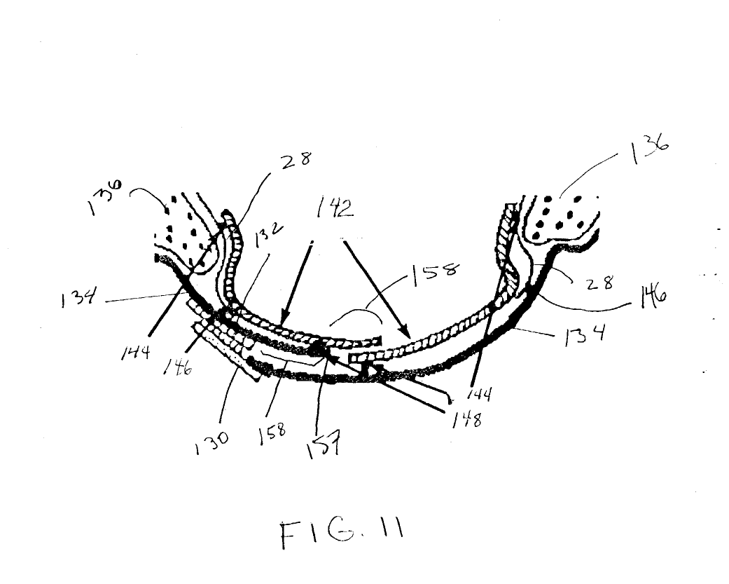

FIG. 11 is a close-up cross-sectional view of the portion of the side area labeled D of FIG. 10. As can be seen in this view, the “bridge”-like materials 142 overlap the central absorbent portions 136 of the absorbent body structure, Each “bridge”-like material 142 additionally overlaps each other in the Z direction. The “bridge”-like materials, such as stretchable coform materials, or stretchable surge management layer materials, are attached to the stretchable liner 28 via adhesive 144. Alternatively it could be attached with ultra sonic or pressure bonding. The stretchable liner 28, can itself be attached to the stretchable outercover/side panels 134 via adhesive 146. Likewise, the “bridge”-like materials 142 can be attached themselves to the stretchable outercover/side panels 134 via adhesive 148. It should be noted, that so as to allow for the overlap of the “bridge”-like materials when the garment is fastened and being worn by a user, a portion of the “bridge”-like material overhangs 158 the lateral most edge 157 of the article chassis. It should be appreciated, however, that such overlap may occur by either of the “bridge”-like materials, depending on the respective placement of the fastening system employed. The illustration of this particular configuration is not meant to be limiting in any way. In the illustrated arrangement of FIG. 11, the loop component of the hook and loop fastening system, is positioned along the outercover/side panels at a location away from the lateral most edge of the chassis ear portions, such that a portion of both the outercover/side panels and the “bridge”-like material overlaps 158 in the Z direction, the adjacent outercover (of the ear portions) when the article is fastened shut. In this fashion, a liquid barrier is created by liquid impermeable outercovers.

It should be appreciated that the materials of the previously described structural components may vary, but may be for example those described earlier herein. For instance, in the case of a “bridge”-like surge material, such a material may be an elastomeric material including styrenic block-copolymers, such as a stretch bonded laminate including a film elastomeric component or a continuous filament strand elastomeric component. Such material may be attached in a stretched state adjacent to one or between two through air-bonded carded webs and then allowed to retract. Such a retraction, as in a stretch-bonded laminate, would cause a gathering of either the one or two outer facing layers. The gathered material would then provide stretchable functionality (up to the point of the opened gathers) and likewise absorbency/wicking functionality.

The facings can be for instance, any nonwoven material, desirably having a basis weight, for example, between about 0.3 and 0.8 osy. In the case of the stretch bonded laminate material described, if one facing is employed, it is desirable that such facing be situated such that it contacts the body of a wearer, rather than the elastomeric component. In this fashion, the possibility of irritation or “red-lining” of the user's skin is reduced. The elastomeric component, may be for example a KRATON styrenic block copolymer compound, extruded at about 4 to about 16 strands per inch and at a basis weight of between about 5 to 20 gsm. More desirably, such an elastomeric component may be extruded at about 8-10 strands per inch and at about 8-12 gsm basis weight. It should be appreciated that this description is meant to be only exemplary, and other elastomeric components can likewise be employed. For instance, prefabricated elastomeric strands may be used, such as LYCRA or the like, polymeric strands.

The “bridge”-like materials are designed to encourage and control the down-hill flow of body exudates to higher capacity absorbent material stored in the back of the absorbent article. Wicking “bridge”-like materials are utilized to wick the moisture around the hips. Without wishing to be bound by theory, it is believed that any overflow of free liquid (that is not absorbed on the front side of the absorbent article) during an insult, would flow over the surface of and wick through the wicking “bridge”-like material to the back absorbent material. In this fashion, the absorbent body structure is prepared to handle numerous product insults.

### EXAMPLE

A hypothetical example of such a surround stretch absorbent article may be the following. This example assumes the article would be aligned along the Y direction shown in FIG. 2 when made in a continuous web process. In other words, it assumes a process where the front of one pant follows the back of a preceding pant in a continuous web or vise versa. If the articles are made in a process where the pants are aligned in the X direction, side by side in the web, or in a hybrid process that combines both front to back and side to side alignment of components in a continuous web, then adjustments might need to be made in the specifications recited in this particular example. These adjustments would be well known to any skilled in the art.

An absorbent article chassis can include a stretchable outercover of a breathable elastomeric film such as polyether block amides available from Atofina Chemical Company of Philadelphia, Pa., under the trade name PEBAX polyether block amide laminated between two facing layers of polypropylene spunbond.

An exemplary laminate would include spunbond layers between about 0.3 and 0.8 osy that have been necked between about 40 and 80 percent (such as around 60 percent) and creped between about 20 and 70 percent, such as around 40 percent. A U.S. Patent which describes such creping process includes U.S. Pat. No. 3,810,280 which is hereby incorporated by reference in their entirety. The stretchable liner may include for example a perforated elastomeric film of between about 7 to 20 gsm basis weight, or a necked and creped polypropylene spunbond nonwoven web of between about 1-2 osy basis weight. Such a web may be necked between about 40 to 70 percent, desirably about 60 percent, and likewise creped between about 20 and 70 percent, desirably about 40 percent. Such a liner may further be treated with a surfactant to improve wettability, such as by treating it with 0.4-0.7% ACHOVEL Base N62 surfactant, available from Uniquma, a division of ICI, having offices in New Castle, Del.

The stretch absorbent component (the entire absorbent body structure including a central absorbent portion and laterally extending wing portions) of such an article, may be comprised of stretch coform absorbent, such as a coform of Kraton G 2740 styrenic block copolymer compound available from Kraton Polymers of Houston, Tex., Sulfatate HJ Pulp available from Rayonier Inc., Fernandina Beach, Fla., and FAVOR SXM 880 particles obtained from Stockhausen, a business having offices in Greensboro, N.C., in approximately a 10/30/60 percent by weight ratio. Such a material would desirably have a basis weight of between about 100 and 800 desirably about 300 gsm basis weight, and be undensified. Additionally, if desired, such an absorbent article could include a single layer surge material, such as surge as previously described having between about 25 and 125 gsm basis weight, desirably about 85 gsm basis weight. Such a surge layer may be for instance a through air bonded carded web. Such an article may likewise include containment flaps and elastomeric waistbands and legbands of stretch bonded laminate materials.

Each of the above materials may be made by known nonwoven material manufacturing processes and constructed into an absorbent article by known manufacturing processes.

Therefore, in summary, in the chassis of a desirable absorbent article of the current invention, the liner and outer cover member 30 are stretchable and at least a portion of the absorbent body structure 32 is stretchable, and includes a portion positioned around the hip areas of the garment, particularly in the lateral or transverse direction X. For example, the outer cover member 30 may be made substantially of an elastomeric material, and the absorbent body structure may include an elastomeric absorbent material.

It should be understood that resort may be had to various other embodiments, modifications, and equivalents to the embodiments of the invention described herein which, after reading the description of the invention herein, may suggest themselves to those skilled in the art without departing from the scope and spirit of the present invention.

What is claimed is:

1. An absorbent article comprising at least a partially stretchable outercover and at least a partially stretchable liner, said absorbent article having a longitudinal direction and longitudinal ends, and a lateral direction and lateral sides, said article further comprising
a front waist region at a first longitudinal end, a back waist region at an opposite longitudinal end, and a crotch region extending longitudinally between said front and back waist regions;
laterally extending elastomeric front and back ear portions defined at opposed lateral sides of said front and back waist regions, wherein said front and back ear portions of said front and back waist regions are stretchable in at least a direction transverse to the longitudinal direction;
an absorbent body structure that is at least partially stretchable, that is situated between said at least partially stretchable outercover and said at least partially stretchable liner, said absorbent body structure extending longitudinally along said crotch region generally between said front waist region and said back waist region, said absorbent body structure further including a central portion and stretchable laterally extending wing portions that extend laterally along said front and back ear portions;
wherein upon folding said article about a transverse fold line at a longitudinal midpoint thereof to form a pant structure, lateral sides of said front and back ear portions join at side seams of said article, such that said laterally extending wing portion edges are proximate to each other.

2. The absorbent article as in claim 1, wherein said central portion and said laterally extending wing portions are contiguous.

3. The absorbent article as in claim 1, wherein said central portion and said laterally extending wing portions are separate but proximate to each other.

4. The absorbent article of claim 3, wherein said article has a Z direction passing through the article, and further wherein said central portion and said laterally extending wing portions overlap one another in the Z direction.

5. The absorbent article as in claim 1, wherein said central portion includes relatively higher capacity absorbent structure, and said laterally extending wing portions include lower capacity absorbent structure relative to the absorbent structure in said central portion.

6. The article as in claim 5 wherein said central portion has a gram/gram absorbent capacity that is at least 10% or greater than said laterally extending wing portions.

7. The absorbent article as in claim 1, wherein said central portion includes relatively lower capacity absorbent materials, and said laterally extending wing portions include higher capacity absorbent materials relative to the absorbent material in said central portion.

8. The article as in claim 7 wherein said central portion has a super absorbent material with a gram/gram absorbent capacity that is at least 10% less than a super absorbent material in said laterally extending wing portions.

9. The absorbent article as in claim 1, wherein said central portion is stretchable.

10. The absorbent article as in claim 1, wherein said absorbent body structure includes more than one layer.

11. The absorbent article of claim 1, wherein said article includes at least one additional absorbent component layer between said partially stretchable outercover and said absorbent body structure.

12. The absorbent article of claim 11 wherein said at least one additional absorbent component layer has relatively higher absorbent capacity than said absorbent body structure..

13. The absorbent article as in claim 1, wherein said ear portions are bonded at said side seams to define said pant structure.

14. The absorbent article of claim 13, wherein said side seams are ultrasonically bonded.

15. The absorbent article as in claim 13, wherein said side seams are tearable for removing the article from a wearer.

16. The absorbent article as in claim 1, wherein said side seams are releasable and re-attachable.

17. The article in claim 16, wherein said re-attachable side seams are over lain by an extension of said laterally extending wing portions.

18. The article in claim 16, wherein said re-attachable side seams are over lain by bridge-like absorbent materials.

19. The absorbent article of claim 13, wherein said side seams are water impervious.

20. The absorbent article of claim 13 wherein said side seam bonds are through said laterally extending wing portions.

21. The absorbent article of claim 13 wherein said side seam bonds are through said outercover and said liner.

22. The absorbent article of claim 1 wherein said partially stretchable outercover is at least partially elastomeric.

23. The absorbent article of claim 1 wherein said partially stretchable liner is at least partially elastomeric.

24. The absorbent article of claim 1 wherein said laterally extending wing portions are elastomeric.
